(19) United States
(12) Patent Application Publication
(10) Pub. No.: US 2026/0166129 A1
(43) Pub. Date: Jun. 18, 2026

(54) Cure for Thyroid Cancer

(71) Applicant: Brian Haney, Reno, NV (US)

(72) Inventor: Brian Haney, Reno, NV (US)

(21) Appl. No.: 18/981,485

(22) Filed: Dec. 14, 2024

(51) Int. Cl. A61K 38/24; A61F 2/04

(52) U.S. Cl. CPC A61K 38/24; A61F 2002/046

(57) ABSTRACT

The present disclosure provides methods and a device for curing thyroid cancer. In embodiments, the invention provides mechanisms for curing thyroid cancer by replacing the cancerous thyroid with a synthetic biotechnology, utilizing both methods and an implantable device for direct drug delivery. The device, may be a cylindrical titanium tube, containing a hormone store at a specific triiodothyronine to thyroxine ratio, tailored to the individual patient's needs post-surgical removal of the cancerous thyroid.

### BACKGROUND TO THE INVENTION

### Field of the Invention

The field of the invention rests at the intersection of three broader fields, biotechnology, cancer, and endocrinology. Biotechnology is any new and better machine or mechanism for improving the quality of human life or treating disease. Cancer is a disease characterized by the uncontrolled growth and spread of abnormal cells that can damage tissues and organs, ultimately disrupting normal bodily functions. Endocrinology is a medical branch concerned with endocrine glands and hormones, including the thyroid and thyroid hormones. Specifically, the field of the invention is thyroid hormone delivery to patients with cancer.

### Background Art

The thyroid is a bilobal gland located at the base of the neck in front of the windpipe. The thyroid's functionality, essentially synthesizing thyroid hormone, is meticulously modeled through a feedback loop. The pituitary gland and thyroid communicate instructions for controlling hormone production and stabilization. The system works on an iterative loop where cells in the pituitary gland determine the body's normal hormonal range, known as the set point. In other words, the thyroid produces hormones, which are secreted into the blood and then carried to every tissue in the body. As such, the Thyroid produces a hormonal variety governing the human body's metabolism.

Thyroid cancer originates in the thyroid gland. There are several types of thyroid cancer, with the most common being papillary thyroid cancer, followed by follicular, medullary, and anaplastic thyroid cancer. Papillary and follicular cancers, often referred to as differentiated thyroid cancers, are usually treatable and have a good prognosis, especially when detected early. Medullary thyroid cancer can be hereditary, associated with specific genetic mutations, while anaplastic thyroid cancer is the most aggressive and least common form, with a poorer prognosis.

Papillary Thyroid Cancer (PTC) is the most prevalent form, accounting for about 80% of all thyroid cancers. It tends to grow slowly and often affects younger individuals. PTC has a very good prognosis, with high survival rates, even when it spreads to lymph nodes in the neck. It can occur as a single tumor or multiple small tumors within the thyroid and is known for its ability to form papillae, hence the name.

Follicular Thyroid Cancer (FTC) represents around 10-15% of cases. It's more likely to spread through the bloodstream to distant parts of the body like the lungs or bones, rather than just to the lymph nodes. FTC is more common in older patients and in regions with iodine deficiency. Like PTC, it generally has a favorable outcome if detected early, though it can be more aggressive when it metastasizes.

Medullary Thyroid Cancer (MTC) accounts for 3-4% of thyroid cancers. It originates from the C cells of the thyroid which produce calcitonin, a hormone involved in calcium regulation. MTC can be sporadic or hereditary, linked to genetic mutations like RET proto-oncogene in conditions such as Multiple Endocrine Neoplasia type 2 (MEN2). This type's prognosis varies; early detection is crucial as it can spread to lymph nodes early in its course.

Anaplastic Thyroid Cancer (ATC) is the rarest and most aggressive form, making up less than 2% of thyroid cancers. It occurs primarily in older individuals and grows very quickly, often presenting as a rapidly enlarging neck mass. ATC is notoriously difficult to treat due to its aggressive nature and resistance to most therapies, leading to a very poor prognosis with low survival rates.

The symptoms of thyroid cancer can be subtle or nonspecific, which often leads to late diagnosis. Common signs include a lump or swelling in the neck, changes in voice, difficulty swallowing or breathing, and persistent hoarseness. However, many individuals might not experience any symptoms, and cancer is discovered incidentally during routine medical examinations or imaging tests for other conditions. Risk factors include exposure to high levels of radiation, particularly in childhood, a family history of thyroid disease, or certain inherited genetic syndromes. Women are more likely than men to develop thyroid cancer, and the incidence increases with age, although thyroid cancer can occur at any age.

Diagnosis of thyroid cancer typically involves a physical examination, blood tests to check thyroid function, and imaging studies like ultrasound to examine the thyroid gland's structure. If a nodule is detected, a fine needle aspiration biopsy might be performed to gather cells for examination under a microscope. This procedure helps determine if the nodule is cancerous. If cancer is confirmed, further tests like CT scans or MRIs might be used to assess the extent of the cancer's spread. The staging of thyroid cancer helps in planning the treatment strategy, which can vary significantly based on the cancer type, stage, and the patient's overall health.

Current treatment for thyroid cancer generally includes surgery to remove part or all of the thyroid gland, thyroidectomy, followed by radioactive iodine therapy to destroy any remaining thyroid tissue or cancer cells. Hormone replacement therapy is necessary post-surgery to provide the body with the thyroid hormones it needs. In cases of advanced or aggressive cancers, other treatments like external beam radiation therapy or targeted therapies might be employed. The prognosis for most thyroid cancers is very good, with survival rates being high, especially for papillary and follicular types when treated early. Regular follow-up care is essential to monitor for any recurrence, manage side effects from treatments, and adjust hormone levels as needed.

A cure and a treatment are two distinct concepts in the realm of healthcare, often confused with one another due to their overlapping goals but differing in their outcomes and approaches. A cure refers to the complete eradication or reversal of a disease or condition, restoring the individual to a state of health where the disease no longer exists within their body. When a disease is cured, it means that the medical intervention has eliminated the cause of the illness, and the patient is free from both the symptoms and the underlying pathology. For example, antibiotics can cure bacterial infections like strep throat by killing the bacteria responsible for the infection. Cures are ideal because they provide a definitive end to the health issue, eliminating the need for further medical intervention for that specific condition.

On the other hand, a treatment involves measures taken to manage, control, or alleviate symptoms of a disease without necessarily eliminating the disease itself. Treatments aim to improve the quality of life by reducing pain, slowing the progression of the disease, or managing symptoms. For chronic conditions like diabetes or arthritis, treatments can include medications, lifestyle changes, therapies, or surgeries that help manage the condition but do not cure it. The objective of treatment is often to maintain or improve the patient's functionality and comfort over time, rather than to eradicate the disease.

The key difference lies in the outcome and expectation. A cure offers a one-time resolution, while treatment might require ongoing management. For instance, while insulin can be a life-saving treatment for diabetes, it does not cure the disease; it only helps manage blood sugar levels. Moreover, some diseases like many forms of cancer might have treatments that can lead to long-term remission, which can sometimes be considered functionally equivalent to a cure if the disease does not return, but technically, the distinction remains that treatments manage or control, whereas cures eliminate.

The synthetic thyroid is a cylindrical thyroid replica, a biotechnology that reverse engineers the functionality of the human thyroid. Simplicity is truth. The thyroid is a gland in the body that produces the thyroid hormones triiodothyronine and thyroxine. The synthetic thyroid allows for the complete replacement of thyroid and a clean cure.

Synthetic biology is a burgeoning field that harnesses the principles of engineering to redesign organisms or biological systems for specific functions. It combines disciplines like genetics, molecular biology, and computer science to create new biological parts, devices, or systems, or to re-engineer existing ones. In the context of reverse engineering human organs, glands, and other parts, synthetic biology provides tools to understand and mimic the complex structures and functions of human biology.

By studying these natural systems, scientists can attempt to replicate or enhance organ functionality using synthetic components. This includes designing organoids, which are miniaturized, simplified versions of organs grown in vitro, to study development, disease, and drug responses without the need for whole-animal models. Techniques such as gene editing with CRISPR-Cas9 allow for precise modifications to cellular DNA, potentially correcting genetic defects or introducing new functions to cells used in organ construction.

Reverse engineering human organs also involves understanding the intricate feedback loops and cell-to-cell communication that govern organ function. This approach has been extended to glands and other anatomical parts, where synthetic biology can be used to create models that simulate hormone production or other biochemical processes. For instance, researchers are exploring the creation of synthetic endocrine glands capable of autonomously regulating hormone levels. The process often starts with organ-on-chip technology, where microfluidic devices mimic organ-level physiology, allowing for the study of how organs interact with each other and respond to various stimuli. This technology, combined with advances in 3D bioprinting and tissue engineering, aims to one day produce fully functional synthetic organs for transplantation or to serve as external support systems for those with organ failure. However, while these technologies show promise, they also face significant challenges, including ensuring immunocompatibility, achieving the complexity of natural tissues, and navigating ethical and regulatory landscapes.

Reinforcement learning type of artificial intelligence dedicated toward control optimization and intelligent decision making. describes a process by which machines learn to achieve goals. The process involves building models and developing systems for decision making, which are embedded in software programs. Reinforcement learning has been the state-of-the-art in intelligent machine technology since the 1980s. In fact, the two most significant reinforcement learning models formalize the technical process by which machines are programmed to achieve goals. As such, reinforcement learning algorithms are particularly well suited for making decisions under uncertainty.

Today, Markov models serve as the foundation for reinforcement learning systems. Technically, Markov models describe the intelligent decision calculus for partially observable dynamic systems. Reinforcement learning algorithms contain three elements: (1) Model: the description of the agent-environment relationship; (2) Reward: the agent's goal; and (3) Policy: the way in which the agent makes decisions. The goal of reinforcement learning is to identify and select the policy which maximizes expected reward for an agent acting in an environment. In the context of thyroid hormone delivery, the optimal policy would correspond with the optimal patient thyroid hormone homeostasis.

Thyroid hormone synthesis is a three-step process. First, the hypothalamus produces Thyroid Releasing Hormone (TRS), stimulating the pituitary gland to release Thyroid Stimulating Hormone (TSH), Thyrotropin, which in turn activates the thyroid gland. Second, the thyroid gland excretes Thyroxine (T₄) to the bloodstream. Third, T₄ converts to Triiodothyronine (T₃) through deiodination in peripheral tissues. This synthesis is critical for metabolic control in the human body.

Thyroxine (T₄) converts to the active Triiodothyronine (T₃) within cells and peripheral tissues by deiodinases. As such, in contrast to Thyroxine (T₄), the Triiodothyronine (T₃) molecule contains three iodine atoms. Triiodothyronine (T₃) is the physiologically active thyroid hormone. It controls myocardium properties, heart rate, and vascular function. In fact, Triiodothyronine (T₃) affects almost every process in the body. Some suggest the thyroid gland produces T₃ directly. Although, thyroid disease is typically not treated with Triiodothyronine (T₃) supplementation. However, some speculate a Thyroxine (T₄) and Triiodothyronine (T₃) combination might be better. Molecular structures are important because clinical effects resulting from thyroid hormone imbalance are observable at the cellular level.

As such, Thyroid hormone maintenance is extremely critical for adult metabolic activity, and thyroid hormone abnormalities in adolescence can have catastrophic consequences. Thyroid hormone imbalance can have profound effects on the central nervous system. Interestingly, Thyroid hormone receptors are located throughout the brain, highlighting their importance in central nervous system's development and function. Indeed, Triiodothyronine (T₃) and Thyroxine (T₄) also provide feedback to the brain and anterior pituitary gland to regulate thyroid hormone.

Thyroid hormone replacement therapy is a chronic and lifetime endeavor for treatment. Thyroid hormone dosage must be established for each patient individually. Usually, the initial dose is small, with amounts increasing gradually until clinical evaluation and laboratory tests indicate optimal response. The dose required to maintain this response is then continued. It is vital to patients' physical and mental health that thyroid hormone treatment have the correct dosage. Under-treatment with thyroid hormone can have deleterious and disastrous consequences including neurochemical imbalances, depression, and fatigue.

Direct drug delivery devices represent a transformative advancement in the field of medicine, designed to deliver therapeutic agents precisely to targeted areas within the body. These devices aim to enhance the efficacy and safety of treatments by optimizing the concentration of drugs at specific sites, thereby minimizing systemic exposure and reducing side effects. The evolution of these devices has been driven by the need to address limitations associated with traditional drug delivery methods, such as oral ingestion or intravenous injection, which often result in suboptimal drug distribution and a higher likelihood of adverse reactions.

The concept of direct drug delivery can be traced back to early attempts to localize treatment for certain conditions, such as the use of topical creams for skin diseases or eye drops for ocular conditions. However, significant technological advancements over the past few decades have revolutionized this field. Innovations such as micro- and nanotechnology, smart polymers, and bioengineered materials have enabled the development of sophisticated delivery systems that can navigate the complex biological environment of the human body. These devices can be designed to respond to specific stimuli, such as pH changes, temperature variations, or the presence of certain enzymes, ensuring that the drug is released only at the desired location and at the right time.

Examples of direct drug delivery devices include implantable pumps, drug-eluting stents, and targeted nanoparticles. Implantable pumps can be used to administer pain medication or insulin continuously and directly to a specific site, ensuring consistent therapeutic levels. Drug-eluting stents, commonly used in cardiovascular treatments, release drugs slowly to prevent artery blockage post-surgery. Targeted nanoparticles can deliver chemotherapy agents directly to cancer cells, sparing healthy tissue and reducing side effects. These advancements highlight the potential of direct drug delivery devices to improve patient outcomes significantly, providing more effective and personalized therapeutic options for a wide range of medical conditions.

There are three major problems with current thyroid cancer treatment: (1) side effects of treatment, (2) long-term monitoring and management, and (3) quality of life complications.

Thyroid cancer treatments, particularly surgery, radioactive iodine (RAI) therapy, and hormone replacement, can lead to numerous side effects. After thyroidectomy (surgical removal of the thyroid), patients must take thyroid hormone replacement for life, which can be tricky to balance. Hypothyroidism or hyperthyroidism can result if the dosage isn't correctly managed, leading to symptoms like fatigue, weight changes, mood swings, and heart issues. RAI therapy can cause salivary gland damage, temporary or permanent changes in taste, and dry mouth. Radiation and some targeted therapies can also lead to more severe side effects like heart, lung, or kidney damage, especially in older patients or those with pre-existing conditions.

Even after successful treatment, thyroid cancer requires lifelong monitoring. This involves regular blood tests to check thyroid hormone levels, ultrasound scans, or other imaging to look for signs of recurrence. The psychological toll of constant vigilance for cancer return can be significant, leading to anxiety and stress. Moreover, the management of hormone levels post-thyroidectomy requires ongoing adjustments, which can be challenging as individual needs change over time due to factors like aging, other health conditions, or medications.

Treatment for thyroid cancer can impact quality of life in various ways. Surgical complications might include vocal cord paralysis leading to voice changes or permanent hoarseness, hypoparathyroidism due to inadvertent removal or damage to the parathyroid glands, causing low calcium levels and symptoms like muscle cramps or tingling. The emotional and physical toll of dealing with cancer, coupled with the potential for chronic conditions like fatigue or altered metabolism, can significantly affect a patient's daily life, productivity, and mental health. Additionally, for patients with advanced or aggressive forms like anaplastic thyroid cancer, the challenge is even greater due to limited treatment options and poor prognosis, often leading to palliative care considerations rather than curative treatments.

Thus, there exists a need for new medical methods for treating or completely curing patients with thyroid cancer. The disclosed invention solves these problems, providing mechanisms for curing patients with thyroid cancer by simply removing the cancerous thyroid and replacing it with a synthetic thyroid biotechnology.

### SUMMARY OF THE INVENTION

The invention is mechanisms for curing thyroid cancer by replacing a cancerous thyroid with a synthetic thyroid. The mechanisms include methods and a device for direct drug delivery. The methods include distributing thyroid hormone on a timed basis to a patient. The device is implantable with a thyroid hormone store and pump to deliver the hormone to the patient. The mechanisms work together to optimize patient metabolic homeostasis and treat hypothyroidism.

In embodiments, the disclosure is a method for curing thyroid cancer involves signaling the delivery of thyroid hormones on a timed basis, where the timing is set to a twelve-hour interval. This method includes sending a command to a thyroid hormone pump, which then loads a portion of the thyroid hormone supply into a secretion chamber. Subsequently, the thyroid hormone pump transfers the hormones from the secretion chamber into the human body, with the ultimate goal of optimizing metabolic homeostasis in the human body.

In embodiments, the disclosure is a method for curing thyroid cancer involves several key steps. First, the patient is diagnosed with thyroid cancer. Following the diagnosis, a synthetic thyroid is customized and engineered to meet the specific thyroid hormone needs of the patient. The next step is the surgical removal of the cancerous thyroid. After removal, the cancerous thyroid is replaced with the customized and engineered synthetic thyroid. The final step in this method is the optimization of thyroid hormone homeostasis to ensure the patient's health and well-being.

In embodiments, the disclosure is a device designed for curing thyroid cancer consists of a cylindrical titanium tube with several integral components. Inside this tube, there is a thyroid hormone store containing thyroid hormone at a ratio of one part triiodothyronine to thirteen parts thyroxine. The cylindrical tube also includes a pump that administers the thyroid hormone at this same ratio. This device is specifically engineered and customized to meet the individual needs of a patient whose cancerous biological thyroid has been completely removed. Additionally, the cylindrical tube is equipped with a delivery device that ensures the thyroid hormone is pumped into the body at the specified ratio, thereby optimizing metabolic homeostasis.

In certain embodiments, the present disclosure may include the use of certain artificial intelligence technologies to control drug delivery. For example, embodiments may use reinforcement learning or deep reinforcement learning to optimize device and delivery control.

### BRIEF DESCRIPTION OF THE DRAWINGS

FIG. 1 is an illustration of a device for thyroid hormone delivery.

FIG. 2 is an illustration for a method of thyroid hormone delivery.

FIG. 3 is an illustration for a method of thyroid hormone delivery.

FIG. 4 is an illustration of a device for thyroid hormone delivery.

### DETAILED DESCRIPTION OF THE INVENTION

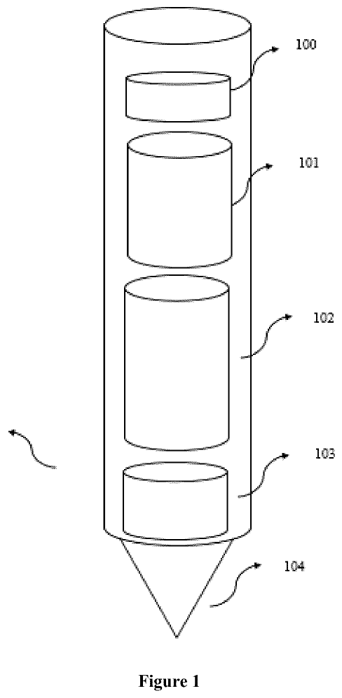

FIG. 1 is an illustration of a device for thyroid hormone delivery. In certain embodiments, a microprocessor 100 sends a signal to a pump 101. The pump creates pressure on a thyroid hormone supply 102, which moves thyroid hormone to a secretion chamber 103. Finally, the thyroid hormone in the secretion chamber moves to a human body via an injection device 104. The complete device operates a synthetic thyroid apparatus 105, replacing or supplementing the functionality of the human thyroid.

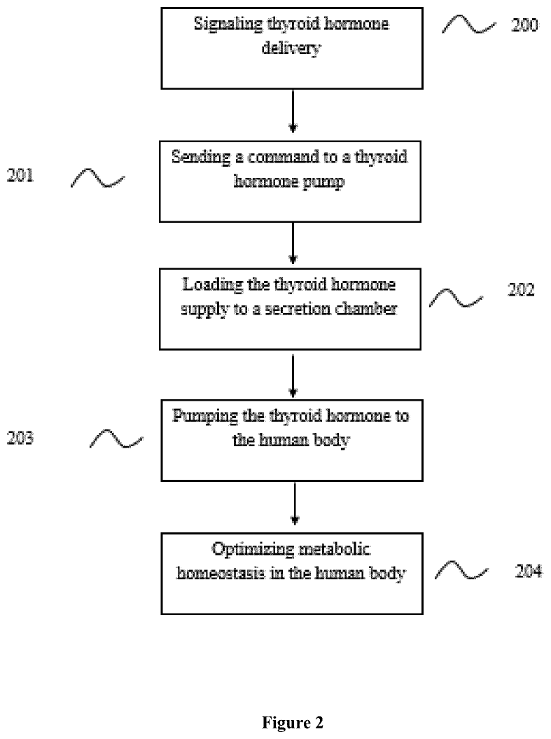

FIG. 2 is an illustration for a method of thyroid hormone delivery. In certain embodiments, the disclosure is a process for thyroid hormone delivery. The process begins with the signaling of thyroid hormone delivery 200. Next, the process proceeds with sending a command of thyroid hormone pump 201. Then, the thyroid hormone supply is loaded to a secretion chamber 202. Next, the thyroid is pumped to the human body 203 to optimize metabolic homeostasis 204.

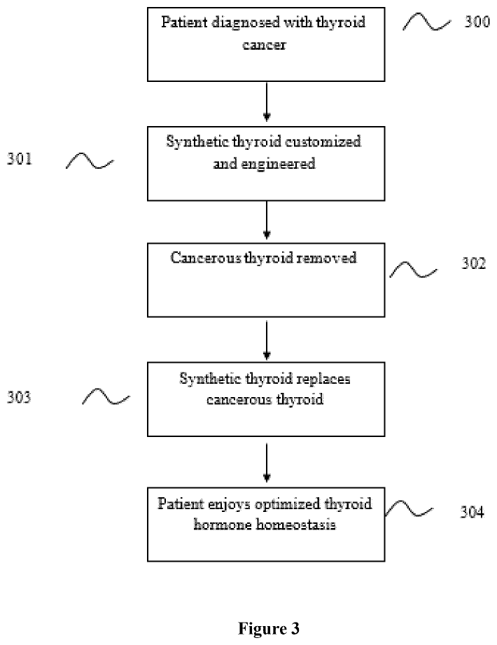

FIG. 3 is an illustration for a method of thyroid hormone delivery. In certain embodiments, the disclosure is a process for replacing a cancerous thyroid with a synthetic thyroid. In such embodiments, a patient is diagnosed with thyroid cancer 300. A synthetic thyroid is customized and engineered for the patient 301. The cancerous thyroid is removed 302. The synthetic thyroid is then implanted, replacing the cancerous thyroid 303. The patient then enjoys the benefits of the synthetic thyroid, which is optimal hormonal homeostasis 304.

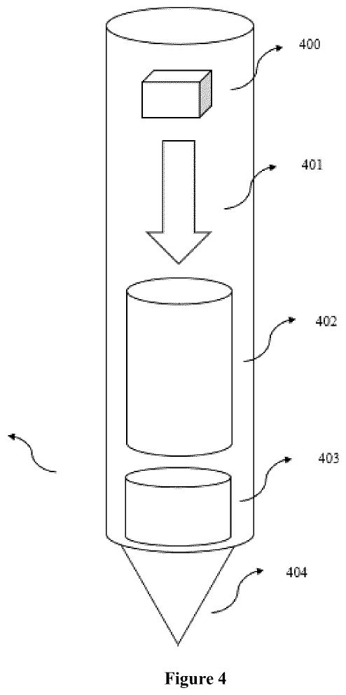

FIG. 4 is an illustration of a device for thyroid hormone delivery. In certain embodiments, a microprocessor 400 sends a signal to 401 to a pump 402. The pump creates pressure on a thyroid hormone supply 403, which moves thyroid hormone to a secretion chamber 403. Finally, the thyroid hormone in the secretion chamber moves to a human body via an injection device 404. The complete device operates a synthetic thyroid apparatus 405, replacing the cancerous human thyroid.

In certain embodiments, the present disclosure is a method for thyroxine delivery. The method may comprise signaling the delivery of the thyroxine on a timed basis. Next, the method may include sending a command to a thyroid hormone pump 101. Then, the method may comprise pumping the thyroxine by a thyroid hormone pump 203. In certain embodiments, the final step may be delivering thyroxine to the human body and maintaining metabolic homeostasis in the human body 304.

In certain embodiments, the disclosure is a method of thyroid hormone delivery. In certain embodiments, the disclosure is a process for replacing a cancerous thyroid with a synthetic thyroid. In such embodiments, a patient is diagnosed with thyroid cancer 300. A synthetic thyroid is customized and engineered for the patient 301. The cancerous thyroid is removed 302. The synthetic thyroid is then implanted 405, replacing the cancerous thyroid 303. The patient then enjoys the benefits of the synthetic thyroid, which is optimal hormonal homeostasis 204.

In certain embodiments, the present disclosure is a method for thyroid hormone delivery. This method involves signaling the delivery of thyroid hormone on a timed basis 200. It includes sending a command to a thyroid hormone pump, which then pumps the thyroid hormone. The thyroid hormone is delivered to the human body through this pump, ultimately maintaining thyroid hormone homeostasis within the body.

In certain embodiments, the present disclosure is a method for delivering thyroid hormones via a synthetic thyroid apparatus 105. This method begins by signaling the delivery of thyroid hormone at specified intervals, ensuring that the timing of hormone administration is carefully controlled. A command is then sent to a thyroid hormone pump, which is responsible for dispensing the hormone 201. The pump activates and releases the thyroid hormone into the human body according to the pre-determined schedule. This precise delivery system ensures that thyroid hormone levels are regulated effectively, thereby optimizing thyroid hormone homeostasis within the body 204.

In certain embodiments, the present disclosure is a method for thyroid hormone delivery comprising several steps. The first step is signaling the delivery of the thyroid hormone on a timed basis 200. The second step is sending a command to a thyroid hormone pump 101. The third step is pumping the thyroid hormone by a thyroid hormone pump 203. The final step is delivering thyroid hormones to the human body and maintaining thyroid hormone homeostasis in the human body 304.

In certain embodiments, the present disclosure is an apparatus for thyroid hormone delivery. The apparatus may be an implantable device with a cylindrical shape. The implantable device may include a microprocessor 100 with embedded instructions for commanding a thyroid hormone pump. The apparatus may also include a thyroid hormone pump, receiving a signal from the microprocessor to pump thyroid hormone supply 102. The apparatus may also include a thyroid hormone supply, the thyroid hormone supply being pumped into the human body by the thyroid hormone pump 203.

In certain embodiments, the present disclosure is an apparatus for thyroid hormone delivery. In such embodiments, the method may comprise an implantable device with a cylindrical shape. The implantable device may further comprise a microprocessor 100 with embedded instructions for commanding a thyroid hormone pump 101. The thyroid hormone pump may receive a signal from the microprocessor to pump thyroid hormone supply 401 from a thyroid hormone supply 102. The thyroid hormone supply may then pumped into the human body at interval times to optimize human metabolic and thyroid hormone homeostasis 204.

In certain embodiments, the present disclosure is an apparatus for thyroid hormone delivery. This apparatus includes an implantable device with a cylindrical shape, designed for efficient integration into the human body. The device is equipped with a microprocessor 100 that contains embedded instructions to control a thyroid hormone pump 101. Upon receiving a signal from the microprocessor, the thyroid hormone pump activates and dispenses the thyroid hormone supply 102. The thyroid hormone supply is then pumped into the human body 203, ensuring consistent and regulated delivery of the hormone as needed.

In certain embodiments, the present disclosure is an apparatus designed for the delivery of thyroid hormones. This apparatus features an implantable device with a cylindrical shape, engineered to be discreetly inserted into the human body. The device houses a microprocessor 100 equipped with embedded instructions that precisely control the operation of a thyroid hormone pump. When the microprocessor sends a command, the thyroid hormone pump activates, drawing from a stored thyroid hormone supply. This supply is then meticulously pumped into the human body, ensuring that the hormone levels are maintained at optimal levels 204. The entire system is designed to provide consistent and regulated hormone delivery, contributing to effective thyroid hormone management and homeostasis.

In certain embodiments, the present disclosure is a method for thyroid hormone delivery. The method may comprise signaling the delivery of the thyroid hormone on a timed basis, wherein the time basis is on a twelve-hour interval. The instruction may be to send a command to a thyroid hormone pump 202, where the thyroid hormone supply is loaded by the pump to a secretion chamber 103 and pumped thyroid hormones from a secretion chamber to the human body by the thyroid hormone pump. The purpose is to optimize metabolic homeostasis in the human body 204.

In certain embodiments, the present disclosure is a method for thyroid hormone delivery. The method may comprise signaling the delivery of the thyroid hormone on a timed basis. The time basis may be a twelve-hour interval. The process may proceed by sending a command to a thyroid hormone pump, loading the thyroid hormone supply by the pump to a secretion chamber, and pumping thyroid hormones from a secretion chamber to the human body by the thyroid hormone pump. The result of the process is optimizing metabolic homeostasis in the human body 204.

In certain embodiments, the present disclosure is a method for thyroid hormone delivery. In such embodiments, the method comprises signaling the delivery of thyroid hormone on a timed basis 200. The time basis may be a twenty-four-hour interval. The process may proceed by sending a command to a thyroid hormone pump, loading the thyroid hormone supply by the pump to a secretion chamber, and pumping thyroid hormones from a secretion chamber to the human body by the thyroid hormone pump. The result of the process is optimizing metabolic homeostasis in the human body 204.

In certain embodiments, the present disclosure is a method for thyroid hormone delivery. In such embodiments, the method comprises signaling the delivery of thyroid hormone on a timed basis 200. The time basis may be an eight-four-hour interval. The process may proceed by sending a command to a thyroid hormone pump. The thyroid hormone may then be loaded by the pump to a secretion chamber 103. The pump may then apply pressure to the thyroid hormone in the secretion chamber, pumping thyroid hormone from a secretion chamber to the human body by the thyroid hormone pump. The result of the process is optimizing metabolic homeostasis in the human body 204.

In certain embodiments, the disclosure is a device for treating hypothyroidism with thyroxine drug administration. The device comprises a sensor measuring thyroxine in the human body using a thyroid hormone sensor, sending the measurement data to a microchip computer processor. The microchip computer processor further comprises an embedded computer program comprising instructions, the instructions provide for calculating optimum levels of thyroxine in the human body, comparing the optimum calculated levels of thyroxine to the measured levels of thyroxine, predicting needed delivery dosage between a range of four micrograms and three-hundred-and-one micrograms, and sending the predicted needed delivery dosage to a second computer program. The second computer program commands drug administration to the human body from a stored thyroid hormone supply, administering thyroxine once every twelve hours, according to the dosage defined by the computer program comprising a set of logical instructions defined by medical experts, helping the patient maintain metabolic homeostasis by measuring and administering thyroid hormone in the human body.

In certain embodiments, the disclosure is a method for treating hypothyroidism with thyroxine drug administration. The method comprises measuring thyroxine in the human body using a thyroid hormone sensor and sending the measurement data to a microchip computer processor. The microchip computer processor further comprises an embedded computer program comprising instructions, defined by medical experts. The computer program calculates optimum levels of thyroxine in the human body, compares the optimum calculated levels of thyroxine to the measured levels of thyroxine, predicts needed delivery dosage between a range of four micrograms and three-hundred-and-one micrograms, and sends the predicted needed delivery dosage to a second computer program. The second computer program commands drug administration to the human body from a stored thyroid hormone supply, administering thyroxine once every twelve hours, according to the dosage defined by the computer program, helping the patient maintain metabolic homeostasis by measuring and administering thyroid hormone in the human body.

In certain embodiments, the disclosure is a method of thyroid hormone delivery. In certain embodiments, the disclosure is a process for replacing a cancerous thyroid with a synthetic thyroid. In such embodiments, a patient is diagnosed with thyroid cancer 300. A synthetic thyroid is customized and engineered for the patient 301. The cancerous thyroid is removed 302. The synthetic thyroid is then implanted, replacing the cancerous thyroid 303. The patient then enjoys the benefits of the synthetic thyroid, which is optimal hormonal homeostasis 304.

In certain embodiments, the disclosure is a method of thyroid hormone delivery. In certain embodiments, the disclosure is a process for replacing a cancerous thyroid with a synthetic thyroid. In such embodiments, a patient is diagnosed with thyroid cancer 300. A synthetic thyroid is customized and engineered for the patient 301. The cancerous thyroid is removed 302. The synthetic thyroid 105 is then implanted 303.

In certain embodiments, the present disclosure is an apparatus for thyroid hormone delivery. This apparatus operates by signaling the delivery of thyroid hormone on a timed basis, with the intervals set to every twelve hours. The system sends a command to a thyroid hormone pump, which then loads the thyroid hormone supply into a secretion chamber. From this chamber, the thyroid hormones are pumped into the human body by the pump. This controlled delivery mechanism ensures the optimization of metabolic homeostasis within the body, maintaining balanced thyroid hormone levels for effective physiological regulation.

In certain embodiments, the present disclosure is an apparatus for thyroid hormone delivery. This apparatus is designed to deliver thyroid hormones through a precise and regulated process. It begins by signaling the delivery of thyroid hormone at set intervals, specifically every twelve hours, ensuring consistent administration. The apparatus sends a command to a thyroid hormone pump, which is integral to the system. Upon receiving this command, the pump loads the thyroid hormone supply into a dedicated secretion chamber. Once the hormone supply is in the chamber, the pump then dispenses the thyroid hormones into the human body. This methodical process is carefully engineered to optimize metabolic homeostasis, ensuring that thyroid hormone levels are maintained within a healthy range and contributing to overall physiological balance.

In certain embodiments, the present disclosure describes a microprocessor-controlled titanium tube designed for the precise delivery of thyroxine into the body. This small, implantable device integrates advanced microelectronics and biocompatible materials to provide an efficient and reliable solution for hormone replacement therapy. At its core, the device features a compact microprocessor programmed to regulate thyroxine release with high precision, utilizing sensors to monitor physiological parameters such as hormone levels and patient activity. This enables real-time feedback and adaptive control, allowing for personalized dosing schedules based on the patient's needs. The thyroxine is encapsulated in a miniature titanium tube, chosen for its excellent biocompatibility, corrosion resistance, and durability, ensuring the hormone remains stable and protected. The tube houses a precision-controlled pump, activated by the microprocessor, which delivers the hormone accurately and consistently into the bloodstream. This pumping mechanism operates silently and efficiently, minimizing patient discomfort while maintaining a steady hormone dose. A long-lasting battery powers the device, designed to minimize power consumption and extend operational life, making it a robust solution for long-term hormone replacement therapy.

In certain embodiments, the present disclosure addresses the efficient absorption of thyroxine once delivered into the body. The thyroxine released from the implant is designed to be bioavailable and readily absorbable by the surrounding tissues. Upon release, thyroxine diffuses through the interstitial fluid and reaches nearby capillaries, entering the bloodstream. The highly vascularized nature of the implantation site ensures rapid uptake of the hormone into the circulatory system.

In such embodiments, once in the bloodstream, thyroxine binds to plasma proteins, primarily thyroxine-binding globulin, which transports it to target tissues throughout the body. The hormone is then absorbed by cells, where it undergoes conversion to its active form, triiodothyronine (T3), within the cells' cytoplasm. This active form interacts with nuclear receptors, initiating the transcription of specific genes that regulate metabolism, growth, and development.

In such embodiments, the controlled release mechanism of the implant ensures a steady and consistent level of thyroxine in the bloodstream, preventing the peaks and troughs associated with conventional oral dosing. This steady state of hormone levels facilitates optimal absorption and utilization by the body's tissues, enhancing the therapeutic efficacy of the treatment and improving overall metabolic stability. By maintaining precise control over thyroxine release and absorption, the implant supports a more balanced and effective management of thyroid hormone levels, ultimately promoting better health outcomes for patients with hypothyroidism.

The pump mechanism in the microprocessor-controlled titanium tube implant offers a precise solution to the burst release problem commonly seen in polymer implants. Unlike traditional polymer matrices that rely solely on passive degradation to release the drug, the pump actively controls the release of thyroxine, allowing for a highly regulated and consistent delivery profile.

Upon implantation, the microprocessor monitors the patient's physiological conditions and determines the optimal release schedule. The pump then dispenses the thyroxine in carefully measured doses, ensuring that the hormone is released steadily over time rather than in an initial burst. This active control mechanism effectively mitigates the risk of an initial surge in hormone levels, which can occur with polymer-only systems as they begin to degrade.

Moreover, the pump's precision in dosage administration allows for fine-tuning of the release rates, which can be adjusted based on real-time feedback from the patient's hormone levels and metabolic needs. This adaptability not only prevents the burst release but also ensures that the patient maintains a stable and therapeutic level of thyroxine, thereby enhancing the efficacy and safety of the treatment. The use of a pump thus represents a significant advancement over traditional polymer implants, providing a reliable and controlled method for hormone delivery.

To make the implant adjustable for dosages, the device can be integrated with a carbon-coded material that interacts with the microprocessor to finely control the release of thyroxine. This advanced material, characterized by its excellent conductivity and responsiveness to electrical signals, allows for precise modulation of the pump's activity based on real-time data. Upon implantation, the microprocessor, embedded within the titanium tube, continuously monitors the patient's physiological parameters through integrated sensors. These sensors can detect fluctuations in thyroid hormone levels, metabolic activity, and other relevant biomarkers. The carbon-coded material is programmed to respond to these signals, adjusting the pump's operation accordingly.

In certain embodiments, the device is made of titanium. The device is approximately 5 mm by 20 mm. The device contains a thyroxine store supply of 20,000 mcg of thyroxine. The device contains a microprocessor with embedded signal software. The signal software triggers a pump within the device to inject 100 mcg of thyroxine to the body every 12 hours.

In embodiments, the present disclosure provides methods and a device for thyroxine delivery. This device is designed for subcutaneous implantation to ensure continuous delivery of thyroxine. It features a reservoir regulated by an embedded microprocessor with advanced signal software. The software triggers a precision pump to release a specific dose of thyroxine at regular intervals. This system enhances patient compliance and maintains optimal metabolic control.

In embodiments, the present disclosure provides methods and a device for thyroxine delivery. This device is designed for subcutaneous implantation to ensure continuous, precise delivery of thyroxine. Constructed from biocompatible titanium, it offers durability and resistance to corrosion, ensuring long-term functionality within the body. The device features an internal reservoir that securely stores thyroxine, maintaining its stability and potency over extended periods. Central to its operation is an embedded microprocessor equipped with advanced signal software, which monitors the device's status and the body's hormone levels through integrated sensors. The microprocessor's software is programmed with sophisticated algorithms to control a miniaturized precision pump, which releases specific doses of thyroxine at regular intervals, mimicking the body's natural hormone release patterns. This timed delivery system enhances patient compliance by providing consistent hormone levels, thereby maintaining optimal metabolic control. The device is powered by a rechargeable battery, which can be replenished via transdermal wireless charging, minimizing the need for invasive maintenance. Safety features, including multiple fail-safes and alerts, ensure reliable operation and notify users or healthcare providers of any malfunctions or low thyroxine levels in the reservoir, making this an efficient and patient-friendly solution for thyroxine delivery.

In certain embodiments, the device is constructed from biocompatible titanium, known for its strength and resistance to corrosion. The device dimensions are approximately 5 mm in width and 20 mm in length, making it small and discreet enough for implantation under the skin. At the core of the device is a reservoir that securely stores 20,000 micrograms (mcg) of thyroxine, a crucial hormone for regulating metabolism. The reservoir is designed to maintain the stability and potency of thyroxine over an extended period, ensuring consistent therapeutic effects. Embedded within the device is a sophisticated microprocessor integrated with advanced signal software. This microprocessor continuously monitors the device's operational status and the body's thyroxine levels through a sensor interface. The signal software is programmed with precise algorithms to ensure accurate dosing.

The device includes a miniaturized, precision-controlled pump mechanism. The pump is connected to the thyroxine reservoir and can deliver exact doses of the hormone. Every 12 hours, the microprocessor triggers the pump to inject 100 mcg of thyroxine into the body. The injection process is carefully controlled to mimic the body's natural hormone release patterns, minimizing any potential side effects or fluctuations in hormone levels. Additionally, the device features a power-efficient battery system that ensures long-term functionality. The battery is rechargeable through transdermal wireless charging, allowing for maintenance without the need for invasive procedures. Safety features include multiple fail-safes and alerts to notify the user or healthcare provider of any malfunctions or low thyroxine levels in the reservoir. Overall, this titanium device provides a reliable, automated solution for the sustained delivery of thyroxine, enhancing patient compliance and maintaining optimal metabolic control.

In certain embodiments, the device is constructed from biocompatible titanium, known for its strength and resistance to corrosion. Measuring approximately 5 mm in width and 20 mm in length, the device is compact and suitable for subcutaneous implantation. It houses a reservoir containing 2 grams of thyroxine, designed to maintain the hormone's stability and potency over an extended period. At the core of the device is a sophisticated microprocessor embedded with advanced signal software. This microprocessor monitors the device's operational status, and through a sensor interface, the body's thyroxine levels. The software is programmed with precise algorithms to ensure accurate dosing. The device includes a miniaturized, precision-controlled pump mechanism connected to the thyroxine reservoir. Every 12 hours, the microprocessor triggers the pump to inject 100 mcg of thyroxine into the body, closely mimicking the body's natural hormone release patterns. Powering this system is a rechargeable battery, which can be replenished through transdermal wireless charging, eliminating the need for invasive maintenance. Safety features include multiple fail-safes and alerts to notify the user or healthcare provider of any malfunctions or low thyroxine levels in the reservoir. Overall, this titanium device offers a reliable, automated solution for sustained thyroxine delivery, enhancing patient compliance and maintaining optimal metabolic control.

In certain embodiments, the disclosure is a method for curing thyroid cancer that involves first diagnosing a patient with thyroid cancer. Following diagnosis, a synthetic thyroid is customized and engineered to match the specific thyroid hormone needs of the patient. The next step is removing the cancerous thyroid. After removal, the cancerous thyroid is replaced with the newly customized and engineered synthetic thyroid. The final step in this method includes optimizing thyroid hormone homeostasis to ensure the patient's metabolic functions are maintained.

In certain embodiments, the disclosure is a method for curing thyroid cancer that begins with diagnosing a patient with the condition. Following this, a synthetic thyroid is customized and engineered to match the patient's specific thyroid hormone needs, with the synthetic thyroid being specifically bioengineered to mimic natural thyroid hormone production patterns. The cancerous thyroid is then removed and replaced with this tailored synthetic thyroid, which continues to optimize thyroid hormone homeostasis by replicating the natural hormone secretion pattern.

In the process of diagnosing thyroid cancer, a synthetic thyroid is engineered to meet the patient's unique hormonal requirements. This synthetic thyroid is programmed to adjust its hormone output based on real-time feedback from the patient's metabolic markers. After removing the cancerous thyroid, the new synthetic thyroid is installed, ensuring that thyroid hormone homeostasis is optimized with dynamic adjustments to match the patient's physiology.

The method for curing thyroid cancer starts with a diagnosis, followed by the customization of a synthetic thyroid that includes self-regulation mechanisms to prevent hormone overproduction or deficiency. After the removal of the cancerous thyroid, this engineered synthetic thyroid is implanted, optimizing thyroid hormone homeostasis through its ability to self-regulate hormone levels.

Diagnosis of thyroid cancer initiates the process where a synthetic thyroid is engineered using biocompatible materials to reduce the risk of immune rejection. This custom thyroid is then placed in the patient after removing the cancerous one, ensuring that thyroid hormone homeostasis is optimized in an environment that is less likely to be rejected by the body.

The method involves diagnosing thyroid cancer and then engineering a synthetic thyroid. The replacement procedure uses a minimally invasive surgical technique to remove the cancerous thyroid, which is then substituted with the custom-engineered synthetic thyroid, all aimed at optimizing thyroid hormone homeostasis with reduced recovery time and fewer complications.

After diagnosing a patient with thyroid cancer, a synthetic thyroid is customized to meet their hormone needs. Post-replacement, the method includes regular monitoring of TSH, T4, and T3 levels to ensure optimal thyroid function, optimizing thyroid hormone homeostasis through vigilant oversight of hormone levels after the cancerous thyroid has been replaced with the synthetic one.

The method for treating thyroid cancer starts with diagnosis and the engineering of a synthetic thyroid that is designed for easy updates or replacements without major surgery due to its modular design. After removing the cancerous thyroid, this synthetic thyroid is implanted, providing a solution for optimizing thyroid hormone homeostasis that can be easily adjusted or upgraded.

Finally, diagnosing thyroid cancer leads to the creation of a synthetic thyroid equipped with sensors for continuous monitoring of hormone levels. This synthetic thyroid is inserted following the removal of the cancerous one, and it optimizes thyroid hormone homeostasis by transmitting data to a device, allowing for precise adjustments by healthcare professionals.

By varying the electrical input to the carbon-coded material, the microprocessor can alter the rate at which the pump dispenses thyroxine. For instance, if the patient's thyroid hormone levels are detected to be lower than the desired threshold, the microprocessor increases the electrical signal to the carbon-coded material, which in turn activates the pump to release a higher dose of thyroxine. Conversely, if hormone levels are within the optimal range, the microprocessor can reduce the signal, slowing down the release rate to maintain steady-state conditions. Additionally, the carbon-coded material can be programmed with specific dosage schedules tailored to the patient's needs. This customization allows for periodic adjustments without the need for invasive procedures. Healthcare providers can remotely update the microprocessor's programming, ensuring that the thyroxine release is continuously optimized for the patient's evolving medical condition. Overall, the integration of carbon-coded material with the microprocessor-controlled pump enables a highly responsive and adaptable hormone delivery system. This approach not only addresses the burst release problem but also provides a scalable solution for personalized medicine, enhancing both the safety and effectiveness of hypothyroidism treatment.

In certain embodiments, the pump for thyroxine in a drug delivery device operates as a critical component ensuring precise and controlled hormone administration. Central to this mechanism is a reservoir that securely stores a concentrated solution of thyroxine, crafted from biocompatible materials to prevent degradation and maintain potency. An embedded microprocessor acts as the control center, utilizing sensors to monitor thyroxine levels in the body, battery status, and the integrity of the pump mechanism. Sophisticated signal software within the microprocessor employs algorithms to regulate the timing and dosage of thyroxine release, mimicking the body's natural hormone release patterns. Upon receiving signals from the software, the precision pump-a miniaturized component capable of delivering exact doses-injects thyroxine at regular intervals. The system also incorporates multiple fail-safes and alert mechanisms to notify users or healthcare providers of any malfunctions or low hormone levels, ensuring reliable and consistent hormone delivery for optimal metabolic control.

In certain embodiments, the present disclosure is a method for thyroid hormone delivery. The method comprises signaling the delivery of the thyroid hormone on a timed basis, wherein the time basis is on a twelve-hour interval. Further, the method includes sending a command to a thyroid hormone pump, loading the thyroid hormone supply by the pump to a secretion chamber, and pumping thyroid hormones from a secretion chamber 403 to the human body by the thyroid hormone pump.

In certain embodiments, the present disclosure is an apparatus for thyroid hormone delivery. In such embodiments, the apparatus is an implantable device with a cylindrical shape 105. The implantable device further comprises a microprocessor 100 with embedded instructions for commanding a thyroid hormone pump. Further the apparatus includes a thyroid hormone pump, receiving a signal from the microprocessor to pump thyroid hormone supply and a thyroid hormone supply 102. Finally, the thyroid hormone supply is pumped into the human body by a thyroid hormone pump.

In certain embodiments, the present disclosure is a method for thyroid hormone delivery. The method comprises signaling the delivery of the thyroid hormone on a timed basis. The method further comprises sending a command to a thyroid hormone pump 201, delivering thyroid hormones to the human body, and optimizing thyroid hormone homeostasis in the human body 204.

A method for curing thyroid cancer involves several steps. First, the delivery of thyroid hormone is signaled on a timed basis, specifically every twelve hours. This triggers a command to be sent to a thyroid hormone pump. The pump then loads a portion of the thyroid hormone supply into a secretion chamber. Following this, the thyroid hormone is pumped from the secretion chamber into the human body by the thyroid hormone pump. This process aims to optimize metabolic homeostasis in the human body.

In this variation of the method for curing thyroid cancer, the thyroid hormone pump is specifically calibrated to load 200 mcg of thyroid hormone into the secretion chamber. This dosage is administered every twelve hours to mimic the natural rhythm of thyroid hormone production. The pump, controlled by advanced software, ensures that this precise amount is pumped into the body, aiming to restore metabolic homeostasis. The 200 mcg dose is particularly suited for patients who require a higher level of thyroid hormone supplementation due to their specific physiological needs or the extent of their thyroid cancer treatment.

Some adjustments to the method involves the thyroid hormone pump loading 50 mcg of thyroid hormone into the secretion chamber. Every twelve hours, this dose is delivered to the patient, providing a consistent supply that helps in managing the metabolic functions disrupted by thyroid cancer. The lower dose of 50 mcg is tailored for those who might be sensitive to higher levels of thyroid hormone or are in the initial stages of post-treatment recovery, ensuring a balanced approach to hormone replacement therapy.

In other versions, the method specifies that 150 mcg of thyroid hormone is loaded into the secretion chamber by the pump. This dosage is administered on a twelve-hour schedule to replicate the body's natural hormone regulation. The 150 mcg dose is designed for patients who need a moderate level of thyroid hormone supplementation, striking a balance between effectiveness and minimizing potential side effects, thereby aiding in the restoration of metabolic balance post-thyroid cancer treatment.

Here, the method involves loading 175 mcg of thyroid hormone into the secretion chamber. This amount is dispensed every twelve hours by the thyroid hormone pump, aiming to maintain optimal metabolic function in patients undergoing thyroid cancer treatment. This dosage level might be indicated for those whose thyroid function requires a slightly higher than average supplement, offering a tailored approach to hormone therapy that supports the body's needs while mitigating the risks of over-supplementation.

In certain embodiments, the disclosure A method for curing thyroid cancer begins with diagnosing a patient with the condition. Following this, a synthetic thyroid is customized and engineered to match the patient's specific thyroid hormone needs, with the synthetic thyroid being specifically bioengineered to mimic natural thyroid hormone production patterns. The cancerous thyroid is then removed and replaced with this tailored synthetic thyroid, which continues to optimize thyroid hormone homeostasis by replicating the natural hormone secretion pattern.

In the process of diagnosing thyroid cancer, a synthetic thyroid is engineered to meet the patient's unique hormonal requirements. This synthetic thyroid 105 is programmed to adjust its hormone output based on real-time feedback from the patient's metabolic markers. After removing the cancerous thyroid, the new synthetic thyroid is installed, ensuring that thyroid hormone homeostasis is optimized with dynamic adjustments to match the patient's physiology.

The method for curing thyroid cancer starts with a diagnosis, followed by the customization of a synthetic thyroid that includes self-regulation mechanisms to prevent hormone overproduction or deficiency. After the removal of the cancerous thyroid, this engineered synthetic thyroid is implanted, optimizing thyroid hormone homeostasis through its ability to self-regulate hormone levels.

Diagnosis of thyroid cancer initiates the process where a synthetic thyroid is engineered using biocompatible materials to reduce the risk of immune rejection. This custom thyroid is then placed in the patient after removing the cancerous one, ensuring that thyroid hormone homeostasis is optimized in an environment that is less likely to be rejected by the body.

The method involves diagnosing thyroid cancer and then engineering a synthetic thyroid. The replacement procedure uses a minimally invasive surgical technique to remove the cancerous thyroid, which is then substituted with the custom-engineered synthetic thyroid, all aimed at optimizing thyroid hormone homeostasis with reduced recovery time and fewer complications.

After diagnosing a patient with thyroid cancer, a synthetic thyroid is customized to meet their hormone needs. Post-replacement, the method includes regular monitoring of TSH, T4, and T3 levels to ensure optimal thyroid function, optimizing thyroid hormone homeostasis through vigilant oversight of hormone levels after the cancerous thyroid has been replaced with the synthetic one.

The method for treating thyroid cancer starts with diagnosis and the engineering of a synthetic thyroid that is designed for easy updates or replacements without major surgery due to its modular design. After removing the cancerous thyroid, this synthetic thyroid is implanted, providing a solution for optimizing thyroid hormone homeostasis that can be easily adjusted or upgraded.

Finally, diagnosing thyroid cancer leads to the creation of a synthetic thyroid equipped with sensors for continuous monitoring of hormone levels. This synthetic thyroid is inserted following the removal of the cancerous one, and it optimizes thyroid hormone homeostasis by transmitting data to a device, allowing for precise adjustments by healthcare professionals.

In certain embodiments, the present disclosure may introduce an innovative control mechanism for the thyroid hormone pump, employing simulation-trained reinforcement machine learning software. This software learns from simulated scenarios and patient data to dynamically adjust the timing and dosage of thyroid hormone delivery. By doing so, it ensures that the hormone levels are optimized for everyone's metabolic requirements, enhancing the efficacy of the cure or treatment for thyroid cancer by providing a personalized and adaptive hormone replacement strategy.

In certain embodiments, the present disclosure introduces a novel control system for thyroid hormone pumps, leveraging a sophisticated simulation-trained reinforcement learning (RL) algorithm known as Proximal Policy Optimization (PPO). PPO is particularly well-suited for this application because it offers a balance between learning efficiency and stability, which are critical when dealing with human health. This algorithm learns from simulated scenarios that replicate various physiological states and patient data, allowing it to predict and adjust the timing and dosage of thyroid hormone delivery dynamically.

The PPO algorithm functions by optimizing a policy through trial and error within these simulations, where it attempts different strategies for hormone administration. Unlike other RL methods, PPO uses a clipped objective function to limit the change in policy during each update, thus preventing large, potentially harmful adjustments to the hormone dosage. This conservative approach ensures that the learning process does not lead to significant deviations from previously successful strategies, which is crucial in medical contexts where patient safety is paramount.

By integrating real patient data, including metrics like thyroid function tests, metabolic rates, and responses to previous treatments, the PPO system refines its policy over time. This continuous learning cycle allows the algorithm to tailor hormone delivery to each patient's unique metabolic needs, enhancing the effectiveness of thyroid cancer treatment or management of other thyroid conditions. The timing of hormone delivery is adjusted based on patterns in daily activities, meal times, and sleep cycles, while the dosage is fine-tuned according to current health status and external influences like stress or illness.

The use of PPO in this context not only personalizes treatment but also potentially increases its efficacy by reducing the risks associated with static dosing protocols. This dynamic adjustment can lead to fewer side effects and better management of thyroid hormone levels, which is vital for patients who have undergone thyroidectomy or are dealing with thyroid dysfunction. However, the implementation of such an advanced system would necessitate extensive clinical trials to validate its performance, safety, and compliance with medical regulations.

Such a control mechanism may use neural networks processing data to optimize release for the thyroid hormone supply. Such data may include the difference between the current and optimal concentration of thyroid hormone, as well as the projected optimal times for release of the drug, and the projected release profile for the hormone and the rate at which it would be distributed throughout the body. For example, various partial derivative calculations may be used to update the neural networks weights through backpropagation.

$$\frac{{\partial }^{2} {x}^{*}}{\partial {l}^{2}} + \frac{{\partial }^{2} {x}^{*}}{\partial {p}^{2}} + \frac{{\partial }^{2} {x}^{*}}{\partial {a}^{2}} = {min}_{{x}^{*}} {x}^{*} ( {x}_{i}^{∘} - {x}_{j}^{∘} ) \qquad ( 1 )$$

$$\frac{{\partial }^{2} {x}^{*}}{\partial {l}^{2}} + \frac{{\partial }^{2} {x}^{*}}{\partial {p}^{2}} + \frac{{\partial }^{2} {x}^{*}}{\partial {a}^{2}} = {min}_{{x}^{*}} {x}^{*} ( {x}_{j}^{∘} - {x}_{i}^{∘} ) \qquad ( 2 )$$

Equation 1 and Equation 2 are examples of partial differential equations, which may be used for backpropagation through neural networks. Backpropagation updates the weights for the neural network to optimize performance.

In certain embodiments, a PPO algorithm works by computing an estimator of the policy gradient and iterating with a stochastic gradient optimization algorithm. This allows for an optimized control algorithm to be simulation trained. The PPO update algorithm may be defined according to Equation 3.

$${θ}_{k + 1} = arg {max}_{θ} {𝔼}_{s , a ∼ {π}_{{θ}_{k}}} [ L ( s , a , {θ}_{k} , θ ) ] . \qquad ( 3 )$$

Here, L(s, a, θₖ, θ) is the objective function, θ are the policy parameters, θₖ are the policy parameters for k experiment. Generally, the PPO update is a method of incremental improvement for a policy's expected return, defined by variables relating to controlled drug delivery.

In certain embodiments, a convolutional neural network may compute an approximation of value for each state-action pair.

$$Q ( s , a ; ϕ ) \approx ( s , a ) . \qquad ( 4 )$$

In Equation 4, φ represents the function parameters, which are a function's variables. This algorithm may be trained on simulation data for the purpose of real-world testing and deployment.

In certain embodiments, the decision algorithm may be determined by a trained agent. The neural network may iterate until the convergence of the Q-function determined by the Bellman Equation.

$${Q}^{*} ( s , a ) = {E}_{{s}^{′} ∼ ε} [ r + γ {max}_{{a}^{′}} {Q}^{*} ( {s}^{′} , {a}^{′} ) \vert s , a ] . \qquad ( 5 )$$

In Equation 5, [symbol] refers to the expectation for all states, r is the reward, γ is a discount factor typically defined 0<γ<1, allowing present rewards to have higher value. Equation 5 defines the optimal Q-function and allows the agent to consider the reward from its present state as greater relative to similar rewards in future states.

In certain embodiments, an optimal policy may pre-trained and developed in a simulation environment. After the optimal policy is defined according to Equation 6.

$${π}^{*} = {Q}^{*} ( {s}^{′} , {a}^{′} ) , \qquad ( 6 )$$

The optimized policy may be embedded in a microprocessor.

It is to be understood that while certain embodiments and examples of the invention are illustrated herein, the invention is not limited to the specific embodiments or forms described and set forth herein. It will be apparent to those skilled in the art that various changes and substitutions may be made without departing from the scope or spirit of the invention and the invention is not considered to be limited to what is shown and described in the specification and the embodiments and examples that are set forth therein. Moreover, several details describing structures and processes that are well-known to those skilled in the art and often associated with biotechnologies are not set forth in the following description to better focus on the various embodiments and novel features of the disclosure of the present invention. One skilled in the art would readily appreciate that such structures and processes are at least inherently in the invention and in the specific embodiments and examples set forth herein.

One skilled in the art will readily appreciate that the present invention is well adapted to carry out the objectives and obtain the ends and advantages mentioned herein as well as those that are inherent in the invention and in the specific embodiments and examples set forth herein. The embodiments, examples, methods, and compositions described or set forth herein are representative of certain preferred embodiments and are intended to be exemplary and not limitations on the scope of the invention. Those skilled in the art will understand that changes to the embodiments, examples, methods and uses set forth herein may be made that will still be encompassed within the scope and spirit of the invention. Indeed, various embodiments and modifications of the described compositions and methods herein which are obvious to those skilled in the art, are intended to be within the scope of the invention disclosed herein. Moreover, although the embodiments of the present invention are described in reference to use in connection with biotechnology, ones of ordinary skill in the art will understand that the principles of the present inventions could be applied to other types of biotechnology for a wide variety of applications.

I claim:

1. A method for curing thyroid cancer, the method comprising:
signaling the delivery of thyroid hormone on a timed basis, wherein the time basis is on a twelve-hour interval,
sending a command to a thyroid hormone pump,
loading a portion of the thyroid hormone supply by the pump to a secretion chamber,
pumping thyroid hormones from a secretion chamber to the human body by the thyroid hormone pump, and
optimizing metabolic homeostasis in the human body.

2. The method of claim 1, wherein the portion of thyroid hormone supply loaded is 200 mcg.

3. The method of claim 1, wherein the portion of thyroid hormone supply loaded is 100 mcg.

4. The method of claim 1, wherein the portion of thyroid hormone supply loaded is 50 mcg.

5. The method of claim 1, wherein the total thyroid hormone supply is 3 g.

6. The method of claim 1, wherein the pump is controlled by a simulation trained reinforcement machine learning software.

7. A method for curing thyroid cancer, the method comprising:
diagnosing a patient with thyroid cancer,
customizing and engineering a synthetic thyroid to the specific thyroid hormone needs of the patient,
removing the cancerous thyroid,
replacing the cancerous thyroid with the customized and engineered synthetic thyroid,
signaling, by the synthetic thyroid, the delivery of thyroid hormone on a timed basis, wherein the time basis is on a twelve-hour interval,
sending a command to a thyroid hormone pump,
loading a portion of the thyroid hormone supply by the pump to a secretion chamber,
pumping thyroid hormones from a secretion chamber to the human body by the thyroid hormone pump, and
optimizing thyroid hormone homeostasis in the patient's body.

8. The method of claim 7, wherein the synthetic thyroid is bioengineered to mimic natural thyroid hormone production patterns.

9. The method of claim 7, wherein the customization of the synthetic thyroid includes programming it to adjust hormone output based on real-time feedback from the patient's metabolic markers.

10. The method of claim 7, wherein the engineered synthetic thyroid incorporates self-regulation mechanisms to prevent hormone overproduction or deficiency.

11. The method of claim 7, wherein the synthetic thyroid is designed with materials that are biocompatible and reduce the risk of immune rejection.

12. The method of claim 7, wherein the replacement procedure involves a minimally invasive surgical technique to enhance recovery and minimize postoperative complications.

13. The method of claim 7, wherein post-replacement monitoring includes regular assessments of TSH, T4, and T3 levels to ensure optimal thyroid function.

14. The method of claim 7, wherein the synthetic thyroid can be updated or replaced without major surgery through a modular design allowing for component upgrades.

15. The method of claim 7, wherein the synthetic thyroid includes sensors for continuous monitoring of hormone levels, transmitting data to a device for adjustment by healthcare professionals.

16. A device for curing thyroid cancer, the device comprising:
a cylindrical titanium tube, further comprising:
a thyroid hormone store, the thyroid hormone store further comprising thyroid hormone at a ratio of parts one to thirteen, triiodothyronine to thyroxine,
the cylindrical tube further comprising a pump, pumping the thyroid hormone at a ratio of parts one to thirteen, triiodothyronine to thyroxine,
the cylindrical tube further being engineered and customized for the specific needs of a patient, having their cancerous biological thyroid completely removed, and
the cylindrical tube further comprising a delivery device by which the thyroid hormone at a ratio of parts one to thirteen, triiodothyronine to thyroxine, is pumped into the body to optimize metabolic homeostasis.

17. The device of claim 16, wherein the titanium tube is coated with a biocompatible material to reduce the risk of rejection by the body.

18. The device of claim 16, wherein the pump operates on a programmable schedule allowing for adjustments based on the patient's metabolic feedback.

19. The device of claim 16, wherein the delivery device includes a diffusion membrane to ensure a steady release of thyroid hormones into the bloodstream.

20. The device of claim 16, wherein the thyroid hormone store has a capacity for at least six months of hormone supply, allowing for less frequent refills or replacements.
